(19) United States
(12) Patent Application Publication
(10) Pub. No.: US 2018/0177969 A1
(43) Pub. Date: Jun. 28, 2018

(54) TREATMENT GAS SUPPLYING APPARATUS

(71) Applicant: NIHON KOHDEN CORPORATION, Shinjuku-ku, Tokyo (JP)

(72) Inventors: Shinji YAMAMORI, Shinjuku-ku, Tokyo (JP); Yoshihiro TAKAYANAGI, Shinjuku-ku, Tokyo (JP); Mitsushi HYOGO, Shinjuku-ku, Tokyo (JP)

(73) Assignee: NIHON KOHDEN CORPORATION, Shinjuku-ku, Tokyo (JP)

(21) Appl. No.: 15/738,911

(22) PCT Filed: Jun. 16, 2016
(86) PCT No.: PCT/JP2016/002913

§ 371 Date: Dec. 21, 2017

(30) Foreign Application Priority Data
Jun. 23, 2015 (JP) 2015-125195

(51) Int. Cl. A61M 16/12; A61M 16/10; A61M 16/00

(52) U.S. Cl. CPC A61M 16/12; A61M 16/1005; A61M 16/0003; A61M 2202/0233; A61M 2202/0266; A61M 2202/025; A61M 2202/0208; A61M 2016/0033; A61M 2202/0225; A61M 2202/0275

(57) ABSTRACT

A treatment gas supplying apparatus includes: a first mixed gas producing section that is configured to produce a first mixed gas in which a first gas and a treatment gas having a treatment effect are mixed with each other in a first ratio; and a second mixed gas producing section that is configured to produce a second mixed gas in which a second gas and the treatment gas are mixed with each other in a second ratio, the second ratio being within a constant difference from the first ratio.

### TECHNICAL FIELD

The present invention relates to an apparatus for supplying a treatment gas (hereinafter, such an apparatus is referred to as a treatment gas supplying apparatus).

### BACKGROUND ART

Recently, it has been reported that gaseous molecules such as nitrogen monoxide (NO), hydrogen sulfide (H₂S), carbon monoxide (CO), or hydrogen (H₂) have various treatment effects (for example, PTL 1, NPL 1, and NPL 2). Usually, such gaseous molecules are mixed with air or oxygen, and then inhaled into the human body. Gaseous molecules are mixed in a ratio of several PPM to several % to air or oxygen.

A mixed gas which is to be administered to the human body is obtained by mixing a treatment gas (such as above-described carbon monoxide) in a constant ratio with air or the like. The mixed gas is compressed and stored in, for example, a gas cylinder. With respect to a spontaneously breathing patient, an apparatus such as a respirator appropriately adjusts the pressure of the gas cylinder, and then causes the mixed gas to be inhaled into the body of the subject.

In the case where treatment is to be applied to a patient who cannot spontaneously breathe, the mixed gas is supplied to a respirator circuit of an artificial respirator. The artificial respirator feeds the mixed gas into the body of the patient through the respirator circuit. Alternatively, a gas cylinder which stores the mixed gas may be connected to an input port of the artificial respirator for compressed air (or compressed oxygen), and the artificial respirator then feeds the mixed gas into the body of the patient through a feeding mechanism.

The above-described treatment gas (gaseous molecules) may be stored in a gas cylinder, or generated by a certain type of generating apparatus. For example, a generator which electrolyzes pure water to generate hydrogen may be disposed in the respirator or the like.

For example, PTL 2 discloses a technique in which such a treatment gas (gaseous molecules) is used. PTL 2 discloses an inhalation method and apparatus for supplying a hydrogen gas into the human body by using the nasal inhalation system.

### CITATION LIST

### Patent Literature

[PTL 1] JP-B-5106110
[PTL 2] JP-A-2005-87257

### Non-Patent Literature

[NPL 1] OSAWA Ikuro, “Molecular Hydrogen Medicine: Current Status and Future Challenges,” Feb. 8, 2011, BIOMEDICAL GERONTOLOGY, 35(1), p. 1-8
[NPL 2] ICHINOSE Fumito et al., “The future of gas mediator,” LiSA VOL. 19, No. 12, p. 1263-1299

### SUMMARY OF INVENTION

### Technical Problem

In treatment of a certain kind of disease, there is a case where the concentration of oxygen in a mixed gas which is to be inhaled is changed (from about 20% to about 100%) in accordance with the condition of the patient. The case where the above-described treatment gas (gaseous molecules) is inhaled by a patient to whom such treatment is applied will be considered. In this case, for example, it may be con-templated that an apparatus is configured by using a gas cylinder of a mixed gas in which the treatment gas is mixed in a predetermined ratio (for example, a mixed gas having a concentration of 2% of the treatment gas), and that of pure oxygen (100% of oxygen). When the concentration of oxygen which is to be inhaled by the patient is changed, however, also the concentration of the treatment gas is changed. When the supply amount from the gas cylinder containing pure oxygen is increased (the oxygen concentration is raised), for example, the concentration of the treatment gas is lowered.

The above-discussed problem occurs not only in the case where the oxygen concentration is changed, but also in the case where a mixed gas containing a treatment gas is further mixed with another gas. In the case where a mixed gas in which the concentration of gaseous molecules other than a treatment gas is changeable is to be produced, namely, there is a problem in that the concentration of the treatment gas in the mixed gas is hardly maintained within a predetermined range.

### Solution to Problem

According to an aspect of the invention, there is provided a treatment gas supplying apparatus comprising: a first mixed gas producing section that is configured to produce a first mixed gas in which a first gas and a treatment gas having a treatment effect are mixed with each other in a first ratio; and a second mixed gas producing section that is configured to produce a second mixed gas in which a second gas and the treatment gas are mixed with each other in a second ratio, the second ratio being within a constant difference from the first ratio.

The first ratio and the second ratio are within a fixed difference. Therefore, the first mixed gas and the second mixed gas contain the treatment gas in a substantially same ratio. Consequently, also the mixed gas which is produced by mixing the first mixed gas with the second mixed gas inside or outside the treatment gas supplying apparatus contains the treatment gas in a predetermined ratio. Even in the case where the concentration of a gas (for example, oxygen) other than the treatment gas is changed in accordance with the object of disease treatment, therefore, the mixed gas which is to be supplied to the patient is in a state where the treatment gas is mixed in a desired ratio.

### Advantageous Effects of Invention

According to the configuration, it is possible to provide a treatment gas supplying apparatus in which, in the case where a mixed gas in which the concentration of gaseous molecules other than a treatment gas is changeable is to be produced, the concentration of the treatment gas in the mixed gas can be maintained within a predetermined range.

### BRIEF DESCRIPTION OF DRAWINGS

FIG. 1 is a block diagram illustrating the configuration of a treatment gas supplying apparatus 1 of Embodiment 1.

FIG. 2 is a block diagram illustrating the configuration of a treatment gas producing section 10 in Embodiment 1.

FIG. 3 is a conceptual view illustrating a concentration change of a mixed gas which is produced by the treatment gas supplying apparatus 1 of Embodiment 1.

FIG. 4 is a block diagram illustrating the configuration of a modification of the treatment gas supplying apparatus 1 of Embodiment 1.

FIG. 5 is a block diagram illustrating the configuration of a treatment gas supplying apparatus 1 of Embodiment 2.

### DESCRIPTION OF EMBODIMENTS

### Embodiment 1

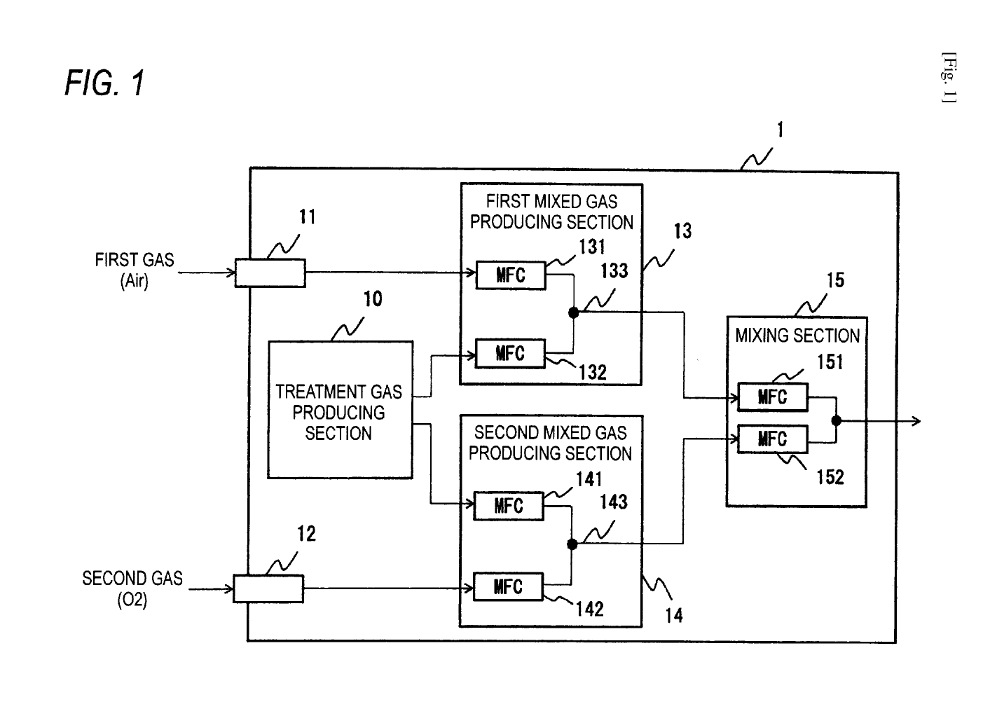

Hereinafter, an embodiment of the invention will be described with reference to the drawings. FIG. 1 is a block diagram illustrating the configuration of a treatment gas supplying apparatus 1 of the embodiment. The treatment gas supplying apparatus 1 has a treatment gas producing section 10, a gas feeding port 11, a gas feeding port 12, a first mixed gas producing section 13, a second mixed gas producing section 14, and a mixing section 15.

A first gas which is an inhalation gas that can be supplied into the body of the patient is fed to the gas feeding port 11. For example, the first gas is one or a mixture of air, oxygen (O₂), nitrogen (N₂), carbon dioxide (CO₂), argon (Ar), and helium (He). In the following description, it is assumed that the first gas is air (about 78% of nitrogen, about 21% of oxygen, etc.).

A second gas which is an inhalation gas that can be supplied into the body of the patient is fed to the gas feeding port 12. The second gas is different in kind from the first gas, and, for example, one or a mixture of air, oxygen (O₂), nitrogen (N₂), carbon dioxide (CO₂), argon (Ar), and helium (He). In the following description, it is assumed that the second gas is pure oxygen (100% of oxygen).

The treatment gas producing section 10 produces a treatment gas inside the treatment gas supplying apparatus 1. The treatment gas is a gas having a treatment effect on the patient. For example, the treatment gas is one or a mixture of hydrogen (H₂), carbon monoxide (CO), hydrogen sulfide (H₂S), and nitrogen monoxide (NO). In the following description, it is assumed that the treatment gas is hydrogen.

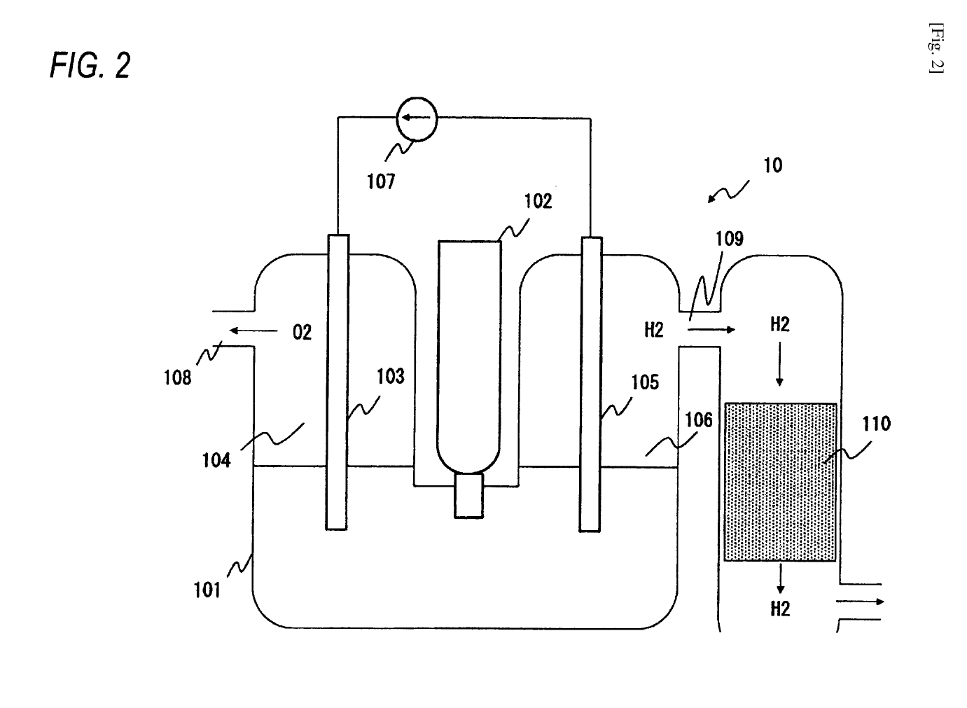

FIG. 2 is a block diagram illustrating an example of the detailed configuration of the treatment gas producing section 10. In the example, the treatment gas producing section 10 electrolyzes water (H₂O) to produce oxygen (O₂) and hydrogen (H₂). In the treatment gas supplying apparatus 1, pure water is supplied from a tank 102 to an electrolytic bath 101. The pure water is supplied to the electrolytic bath 101 in such a manner that the electrolytic bath 101 is not filled with the pure water. The invention is not limited to the configuration in which pure water is stored in a tank. Alternatively, tap water or the like may be stored in a tank, and a mechanism (ion-exchange resin or the like) which removes impurities of the tap water or the like may be disposed.

The electrolytic bath 101 is partitioned into an anode chamber 104 in which an anode 103 is disposed, and a cathode chamber 106 in which a cathode 105 is disposed.

Preferably, each of the anode 103 and the cathode 105 is formed into a long rod-like shape as illustrated, and configured by using a titanium oxide electrode. The titanium oxide electrodes may be formed by powder metallurgy by using, for example, powder of titanium (Ti), titanium oxide (TiO₂), nickel (Ni), iron (Fe), chromium (Cr), or platinum (Pt). Alternatively, each of the titanium oxide electrodes may be formed by powder metallurgy to adsorb titanium (Ti) and titanium oxide (TiO₂) to the periphery of an electrode core member which is formed by a stainless steel rod.

A constant current source 107 is connected to the anode 103 and the cathode 105. The constant current source 107 supplies a current to the anode 103 and the cathode 105. This causes the treatment gas producing section 10 to produce a hydrogen gas (H₂) and an oxygen gas (O₂).

A discharge port 108 from which the oxygen gas is discharged is disposed in an upper portion of the anode chamber 104. The discharge port 108 is requested to com-municate with the exterior of the apparatus. Namely, a configuration where the oxygen gas is discharged to the outside is requested. Of course, a configuration where a mechanism for internally processing the oxygen gas is disposed may be employed.

A discharge port 109 from which the hydrogen gas is discharged is disposed in an upper portion of the cathode chamber 106. As illustrated, the discharge port 109 may be configured so as to be coupled to a gas drying section 110. The gas drying section 110 dries the hydrogen gas discharged from the cathode chamber 106, and then supplies the dried hydrogen gas to the first mixed gas producing section 13 and the second mixed gas producing section 14. The gas drying section 110 may be configured by, for example, silica gel. The configuration illustrated in FIG. 2 is one mode of a configuration which generates a hydrogen gas by electrolysis, and a configuration based on another technique (for example, electrolysis in which different compounds are used) may be employed as far as the configuration can safely generate a hydrogen gas.

Referring again to FIG. 1, hydrogen (treatment gas) is supplied from the treatment gas producing section 10 to the first mixed gas producing section 13 and the second mixed gas producing section 14. The first mixed gas producing section 13 produces a first mixed gas in which air (first gas) and hydrogen (treatment gas) are mixed with each other in a first ratio. The first ratio is a mixture ratio of air and hydrogen, and exhibits a large treatment effect of hydrogen on the patient. For example, the first ratio is 98:2 (i.e., a ratio in which the concentration of hydrogen is 2%). The first ratio may have a certain width. Namely, the first ratio may be a value in a range of, for example, from 97.5 to 98.5:from 2.5 to 1.5. In the case where hydrogen is used as a treatment gas, the first ratio is desired to be within a range of from 99 to 96:from 1 to 4.

The first mixed gas producing section 13 may include a mass flow controller (MFC) 131 and a mass flow controller (MFC) 132. The mass flow controller 131 adjusts the flow rate of air in accordance with the first ratio. Similarly, the mass flow controller 132 adjusts the flow rate of hydrogen in accordance with the first ratio. Air (the output of the mass flow controller 131) and hydrogen (the output of the mass flow controller 132) are mixed with each other in a pipe 133. Therefore, the first mixed gas in which air and hydrogen are mixed in the first ratio is produced. The first mixed gas is supplied to the mixing section 15 through the pipe 133.

The mass flow controller 131 and the mass flow controller 132 are mere examples of a processing section which mixes air and hydrogen with each other. Therefore, the first mixed gas may be produced by using a flow rate controlling system and flow rate detecting system which are based on another method.

The second mixed gas producing section 14 produces a second mixed gas in which oxygen (second gas) and hydrogen (treatment gas) are mixed with each other in a second ratio. The second ratio is a mixture ratio of oxygen and hydrogen, and exhibits a large treatment effect of hydrogen on the patient. The second ratio is within a constant difference from the first ratio, and preferably equal to the first ratio. In the case where the first ratio is 98:2, for example, a ratio which is allowable as the second ratio is from 97.5 to 98.5:from 2.5 to 1.5, preferably 98:2. In the case where the first ratio is from 97.5 to 98.5:from 2.5 to 1.5, for example, a ratio which is allowable as the second ratio is from 97 to 99:from 3 to 1, preferably from 97.5 to 98.5:from 2.5 to 1.5.

The second mixed gas producing section 14 may include a mass flow controller (MFC) 141 and a mass flow controller (MFC) 142. The mass flow controller 141 adjusts the flow rate of hydrogen in accordance with the second ratio. Similarly, the mass flow controller 142 adjusts the flow rate of oxygen in accordance with the second ratio. Hydrogen (the output of the mass flow controller 141) and oxygen (the output of the mass flow controller 142) are mixed with each other in a pipe 143. Therefore, the second mixed gas in which hydrogen and oxygen are mixed in the second ratio is produced. The second mixed gas is supplied to the mixing section 15 through the pipe 143.

The mass flow controller 141 and the mass flow controller 142 are mere examples of a processing section which mixes oxygen and hydrogen with each other. Therefore, the second mixed gas may be produced by using a flow rate controlling system and flow rate detecting system which are based on another method.

The first mixed gas producing section 13 may be configured so as to have a tank which temporarily stores the first mixed gas that has already undergone the gas mixing processes. Similarly, the second mixed gas producing section 14 may be configured so as to have a tank which temporarily stores the second mixed gas that has already undergone the gas mixing processes.

The first mixed gas and the second mixed gas are supplied to the mixing section 15. Here, the first mixed gas is a gas in which hydrogen and air are mixed with each other in the first ratio, and the second mixed gas is a gas in which hydrogen and oxygen are mixed with each other in the second ratio (preferably, the same ratio as the first ratio). The mixing section 15 outputs a mixed gas in which the first mixed gas and the second mixed gas are mixed with each other. As illustrated in the figure, for example, the mixing section 15 has a mass flow controller 151 and a mass flow controller 152, and outputs the mixed gas which is set to a designated oxygen concentration. The oxygen concentration can be set to from about 21% to 98%. The doctor or the like operates an interface (buttons, touch panel, or the like which is not shown) disposed on a housing, in accordance with the disease and condition of the patient, thereby inputting a desired oxygen concentration.

In the case where 30% is designated as the oxygen concentration, for example, the mixing section 15 produces the mixed gas while making the mixture ratio of the first mixed gas larger than that of the second mixed gas. The mixed gas produced by the mixing section 15 will be used as a gas which is to be administered to the patient. For example, the mixing section 15 supplies the mixed gas which is produced from the first mixed gas and the second mixed gas, through a tube to a mask that is fixed to the vicinity of the nose and mouth of the patient.

Before the mixing of the first mixed gas and the second mixed gas, the mixing section 15 may detect the hydrogen concentrations of the first mixed gas and the second mixed gas, and, when an abnormality is detected in at least one of the concentrations, suspend the mixing process. This enables the mixed gas to be supplied more safely, and can avoid a situation in which gas administration is performed in a state where a small treatment effect is exerted on the patient.

The mass flow controller 151 and the mass flow controller 152 are mere examples of a processing section which mixes the first mixed gas and the second mixed gas with each other. Therefore, the mixed gas may be produced by using a flow rate controlling system and flow rate detecting system which are based on another method.

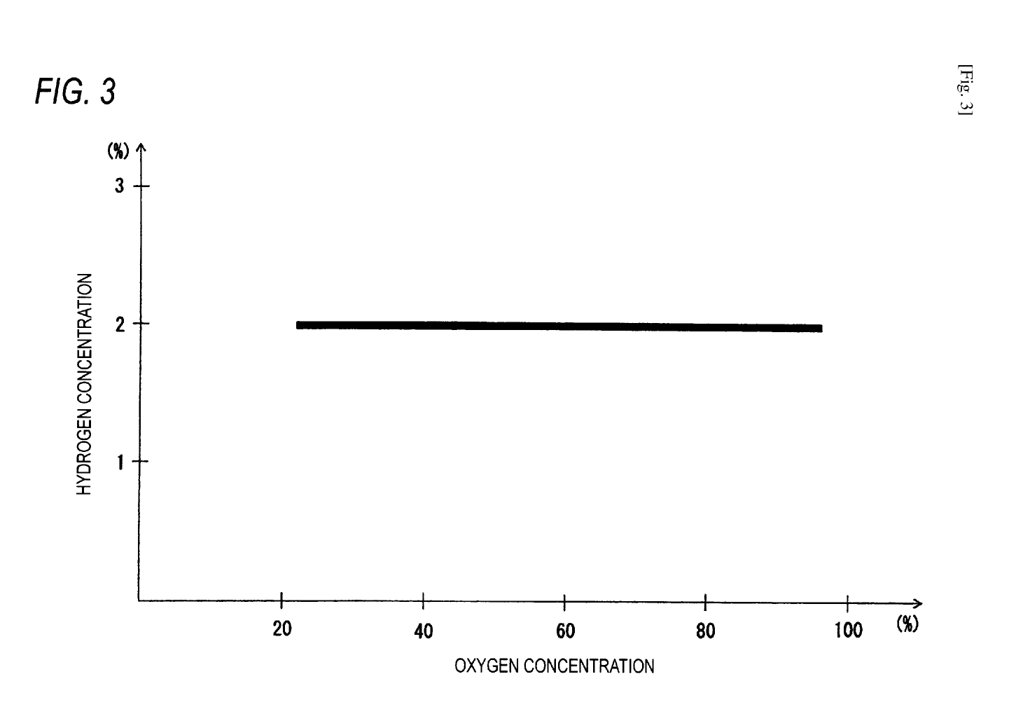

FIG. 3 is a graph showing the composition ratio of the mixed gas (mixed gas which is output from the mixing section 15) produced by the treatment gas supplying apparatus 1 of the embodiment. In the example of FIG. 3, it is assumed that both the first and second ratios are 98:2. Even in the case where the oxygen concentration is changed as illustrated in the figure (the concentrations are controlled by the mass flow controllers 151, 152), the hydrogen concentration remains unchanged from about 2%. Even in the case where the oxygen concentration is changed, namely, the mixed gas can be supplied to the patient without changing the concentration of hydrogen at which a treatment effect is exerted. Therefore, treatment having a large effect can be always applied to the patient.

Then, effects of the treatment gas supplying apparatus 1 of the embodiment will be described. As described above, the first mixed gas producing section 13 produces the first mixed gas in which the first gas and the treatment gas are mixed with each other in the first ratio, and the second mixed gas producing section 14 produces the second mixed gas in which the second gas and the treatment gas are mixed with each other in the second ratio. The first ratio and the second ratio are within a fixed difference (preferably, equal to each other). Therefore, the first mixed gas and the second mixed gas contain the treatment gas in an approximately equal ratio. The first ratio and the second ratio are set to a ratio at which the treatment gas exerts a large treatment effect. Therefore, also the mixed gas which is produced by mixing the first mixed gas and the second mixed gas in the mixing section 15 contains the treatment gas in a desired ratio. Even in the case where the concentration of a gas other than the treatment gas is changed in accordance with the object of disease treatment, therefore, the mixed gas which is to be supplied to the patient is in a state where the treatment gas is mixed in a desired ratio.

For example, the treatment gas is one or a mixture of hydrogen, carbon monoxide, hydrogen sulfide, and nitrogen monoxide. It has been known that all of these gases exert a treatment effect on the patient.

The first gas and the second gas are gases containing one or a mixture of hydrogen, carbon monoxide, hydrogen sulfide, and nitrogen monoxide. When the mixing concentration and the like are not erroneously set, all of these gases can be used as an inhalation gas to the patient, and are less likely to adversely affect the human body.

### Modification

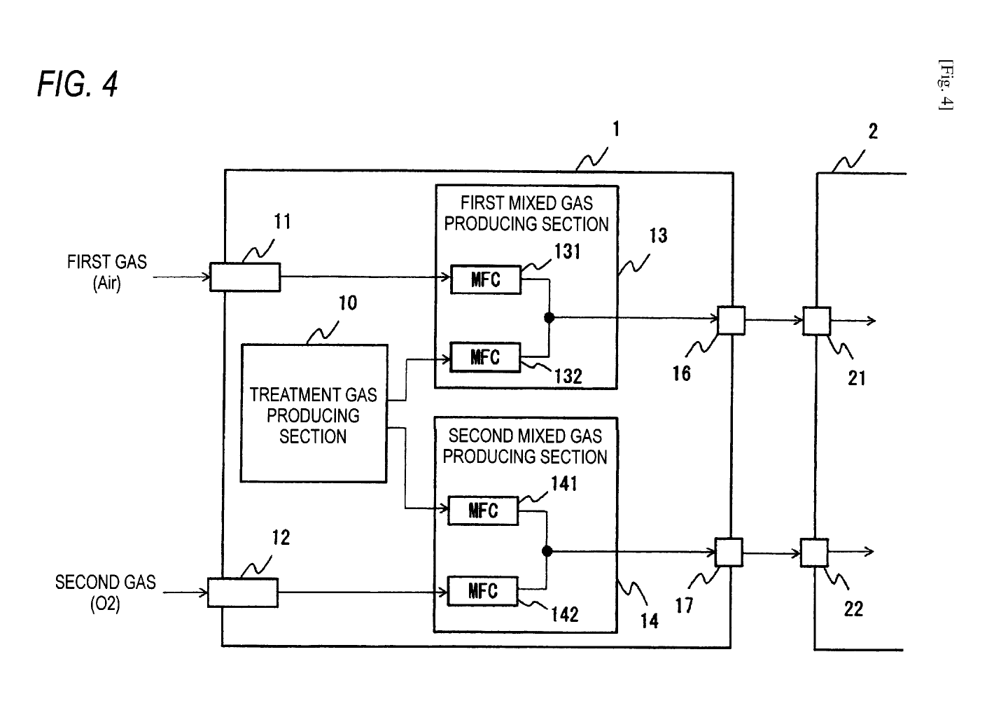

FIG. 4 is a block diagram illustrating a modification of the treatment gas supplying apparatus 1 illustrated in FIG. 1. The treatment gas supplying apparatus 1 of the modification has a configuration which is not provided with the mixing section 15 as compared with the configuration of FIG. 1. The first mixed gas producing section 13 is connected to a first connection port 16 through a pipe (or a tube), and the second mixed gas producing section 14 is connected to a second connection port 17 through a pipe (or a tube).

The first connection port 16 is connected to the first mixed gas producing section 13, and also to a third connection port 21 of an artificial respirator 2. The second connection port 17 is connected to the second mixed gas producing section 14, and also to a fourth connection port 22 of the artificial respirator 2. That is, the first mixed gas producing section 13 supplies the first mixed gas to the artificial respirator 2 through the first connection port 16 and the third connection port 21. Similarly, the second mixed gas producing section 14 supplies the second mixed gas to the artificial respirator 2 through the second connection port 17 and the fourth connection port 22.

The artificial respirator 2 mixes the first mixed gas with the second mixed gas to produce the mixed gas which is to be supplied to the patient. Also in this case, the first mixed gas and the second mixed gas have a desired hydrogen concentration (for example, 2%). In the case where the artificial respirator 2 mixes the first mixed gas with the second mixed gas to produce the mixed gas which is to be administered to the patient, therefore, the hydrogen concentration of the mixed gas is within a constant range (about 2%). Consequently, the artificial respirator 2 can supply the mixed gas in which the treatment gas is mixed in a ratio at which a large treatment effect is exerted, to the patient.

The artificial respirator 2 is one mode of an external apparatus to which the treatment gas supplying apparatus 1 supplies the first mixed gas and the second mixed gas, and may be replaced with an apparatus of another kind. The configuration of connecting the treatment gas supplying apparatus 1 to the artificial respirator 2 is not limited to that of FIG. 4, and they may be connected to each other by another connecting method.

### Embodiment 2

The treatment gas supplying apparatus 1 of the embodiment is characterized in that the apparatus incorporates the first mixed gas and second mixed gas which have already undergone the gas mixing processes, respectively. Hereinafter, the configuration of the treatment gas supplying apparatus 1 of the embodiment will be described. In the following description, the processing sections which are indicated by the same names and reference numerals as those used in Embodiment 1 are identical with those of Embodiment 1 unless particularly described. It is assumed that the first gas is air, the second gas is oxygen, and the treatment gas is hydrogen.

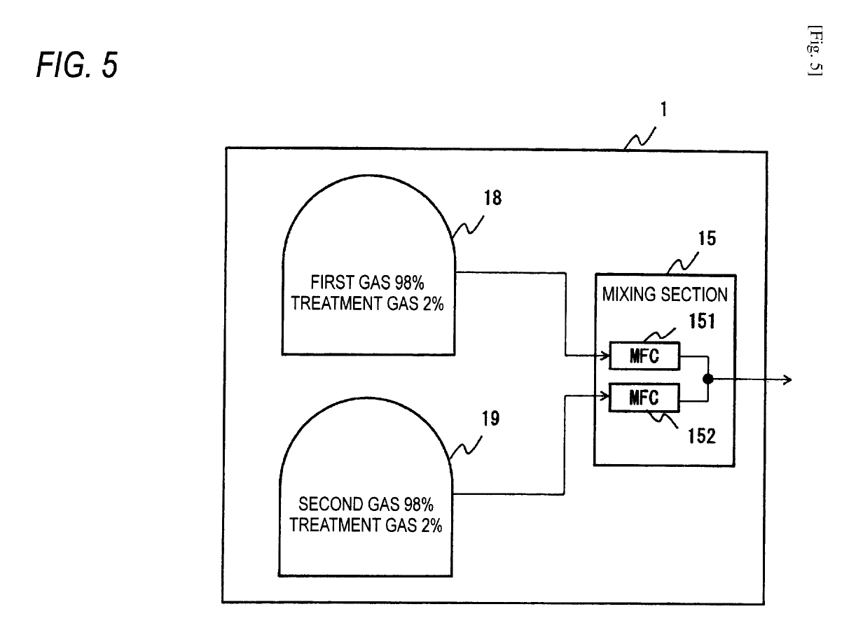

FIG. 5 is a block diagram illustrating the configuration of the treatment gas supplying apparatus 1 of the embodiment. The treatment gas supplying apparatus 1 of the embodiment has a first tank 18 and a second tank 19 in place of the first mixed gas producing section 13 and second mixed gas producing section 14 in the configuration of FIG. 1.

The first tank 18 is a tank which stores the above-described first mixed gas. Namely, the first tank 18 stores the gas which is produced by mixing air (first gas) and hydrogen (treatment gas) with each other in the first ratio. For example, the first tank 18 may be a gas cylinder which stores a compressed medical gas.

Similarly, the second tank 19 is a tank which stores the above-described second mixed gas. Namely, the second tank 19 stores the gas which is produced by mixing oxygen (second gas) and hydrogen (treatment gas) with each other in the second ratio. For example, the second tank 19 may be a gas cylinder which stores a compressed medical gas.

Similarly with Embodiment 1, the mixing section 15 may produce the mixed gas in which the first mixed gas and the second mixed gas are mixed with each other, and ad-minister the mixed gas to the patient. For example, the mixing section 15 may have a configuration incorporating a mass flow controller (MFC) 151 and a mass flow controller 152. The mass flow controller 151 adjusts the flow rate of the first mixed gas, and the mass flow controller 152 adjusts the flow rate of the second mixed gas. Therefore, the oxygen concentration of the mixed gas which is output from the mixing section 15 is adjusted.

Also in the above-described configuration, the hydrogen concentration (concentration of the treatment gas) of the mixed gas which is output from the mixing section 15 is within a constant range (see FIG. 3). Even in the case where the oxygen concentration is changed in accordance with the object of disease treatment, therefore, the mixed gas which is to be supplied to the patient is in a state where hydrogen is mixed in a desired ratio.

Although the invention conducted by the inventor has been specifically described based on the embodiments, the invention is not limited to the above-described embodiments, and it is a matter of course that various changes can be made without departing from the spirit of the invention.

Finally, the hardware configuration and the like of the treatment gas supplying apparatus 1 will be briefly described. Processes such as the controls of the mass flow controllers are realized by various electric circuits and software programs. That is, the treatment gas supplying apparatus 1 is configured so as to internally have storage devices (a primary storage device such as a cache memory, and a secondary storage device such as a hard disk drive), a CPU (Central Processing Unit), and the like.

The present application is based on Japanese Patent Application No. 2015-125195 filed on Jun. 23, 2015, the contents of which are incorporated herein by way of reference.

### INDUSTRIAL APPLICABILITY

According to the invention, it is possible to provide a treatment gas supplying apparatus in which, in the case where a mixed gas in which the concentration of gaseous molecules other than a treatment gas is changeable is to be produced, the concentration of the treatment gas in the mixed gas can be maintained within a predetermined range.

1. A treatment gas supplying apparatus comprising:
a first mixed gas producing section that is configured to produce a first mixed gas in which a first gas and a treatment gas having a treatment effect are mixed with each other in a first ratio; and
a second mixed gas producing section that is configured to produce a second mixed gas in which a second gas and the treatment gas are mixed with each other in a second ratio, the second ratio being within a constant difference from the first ratio.

2. The treatment gas supplying apparatus according to claim 1, further comprising:
a mixing section that is configured to produce a mixed gas in which the first mixed gas and the second mixed gas are mixed with each other.

3. The treatment gas supplying apparatus according to claim 1, further comprising:
a first connection port through which the first mixed gas is supplied to an artificial respirator, and a second connection port through which the second mixed gas is supplied to the artificial respirator.

4. The treatment gas supplying apparatus according to claim 1, wherein
the treatment gas is one or a mixture of hydrogen, carbon monoxide, hydrogen sulfide, and nitrogen monoxide.

5. The treatment gas supplying apparatus according to claim 1, wherein
the first gas is one or a mixture of air, oxygen, nitrogen, carbon dioxide, argon, and helium, and
the second gas is different in kind from the first gas, and one or a mixture of air, oxygen, nitrogen, carbon dioxide, argon, and helium.

6. The treatment gas supplying apparatus according to claim 1, wherein
the treatment gas is hydrogen, and
the treatment gas supplying apparatus further comprises a treatment gas producing section that is configured to electrolyze water to produce the treatment gas.

7. The treatment gas supplying apparatus according to claim 1, wherein
the first ratio is equal to the second ratio.

8. The treatment gas supplying apparatus according to claim 2, wherein,
before the first mixed gas and the second mixed gas are mixed with each other, the mixing section is configured to detect concentrations of the treatment gas in the first mixed gas and the second mixed gas, and, when an abnormality is detected in at least one of the concentrations, suspend the mixing process.

9. (canceled)

10. The treatment gas supplying apparatus according to claim 1, wherein,
the first mixed gas producing section is configured to adjust a flow rate of the first gas and a flow rate of the treatment gas in accordance with the first ratio to produce the first mixed gas.

11. The treatment gas supplying apparatus according to claim 1, wherein,
the second mixed gas producing section is configured to adjust a flow rate of the second gas and a flow rate of the treatment gas in accordance with the second ratio to produce the second mixed gas.

12. The treatment gas supplying apparatus according to claim 1, wherein,
in the first ratio, a ratio of the first gas and the treatment gas is within a range from 99 to 96:from 1 to 4.

13. The treatment gas supplying apparatus according to claim 2, wherein,
a concentration of the second gas can be set to produce the mixed gas.

14. The treatment gas supplying apparatus according to claim 2, wherein,
a concentration of the treatment gas in the mixed gas is within a constant range.

15. A treatment gas supplying apparatus comprising:
a first tank that is configured to store a first mixed gas in which a first gas and a treatment gas having a treatment effect are mixed with each other in a first ratio;
a second tank that is configured to store a second mixed gas in which a second gas and the treatment gas are mixed with each other in a second ratio, the second ratio being within a constant difference from the first ratio; and
a mixing section that is configured to produce a mixed gas in which the first mixed gas and the second mixed gas are mixed with each other.
